(19) United States
(12) Patent Application Publication
(10) Pub. No.: US 2026/0160758 A1
(43) Pub. Date: Jun. 11, 2026

(54) Sulfonate-Containing Water-Solubilizing Peptides

(71) Applicant: Becton, Dickinson and Company, Franklin Lakes, NJ (US)

(72) Inventor: Glenn P. Bartholomew, San Marcos, CA (US)

(21) Appl. No.: 19/124,757

(22) PCT Filed: Dec. 7, 2023
(86) PCT No.: PCT/US2023/082854

§ 371 Date: Apr. 25, 2025

Related U.S. Application Data
(60) Provisional application No. 63/432,122, filed on Dec. 13, 2022.

(51) Int. Cl. G01N 33/542; C07K 17/08; C09B 62/78; C09K 11/06

(52) U.S. Cl. CPC G01N 33/542; C07K 17/08; C09B 62/78; C09K 11/06

(57) ABSTRACT

Sulfonate-containing water-solubilizing peptides are provided. Such water-solubilizing peptides can be attached to, for example, a dye or a tandem dye to increase the solubility thereof. Also provided are methods of using the dyes that include the water-solubilizing peptides, as well as kits that include such dyes.

### CROSS-REFERENCE TO RELATED APPLICATION

This application claims priority to the filing date of U.S. Provisional Patent Application Ser. No. 63/432,122 filed on Dec. 13, 2022; the disclosure of which application is incorporated herein by reference.

### INTRODUCTION

Polyethylene glycol (PEG) groups have been attached to small molecules, nucleotides, peptides, proteins, liposomes, and nanoparticles to improve solubility, stability, and pharmacokinetic properties (Chen et al., ACS Nano, 2021, 15, 14022). In some cases, polymer-drug conjugates have been formed that employ PEG, such as those PEG groups with molecular weights of 0.3 to 60 kDa per polymer (Kong et al., Frontiers in Bioengineering and Biotechnology, 2022, 10, 879988). PEG has also been used in bioconjugation and nanomedicine to prolong blood circulation time and improve drug efficacy (Thi et al., Polymers, 2020, 12, 298).

However, some naive individuals can have pre-existing antibodies that can bind to PEG and induce an immune response (Chen et al., above). At least 25 different pharmaceutical drugs or compositions include PEG groups, including the mRNA-1273 vaccine for COVID-19 that was produced by Moderna, Inc. (Id.). In addition, treating subjects with drugs containing PEG groups has been observed to cause the development of anti-PEG antibodies, which can be undesirable (Thi et al., above).

Additionally, many common methods of synthesizing PEG and other polymers generate polydisperse compositions with broad distributions of molecular weights. Such polydispersity can disadvantageously inhibit control over the properties of the polymers.

### SUMMARY

Sulfonate-containing water-solubilizing peptides are provided. Such water-solubilizing peptides can be attached to, for example, a dye or a tandem dye to increase the solubility thereof. Also provided are methods of using the dyes that include the water-solubilizing peptides, as well as kits that include such dyes.

### BRIEF DESCRIPTION OF THE FIGURES

FIG. 1A shows a schematic representation of a dye with a fluorophore and a water solubilizing peptide that is linked to a biomolecule.

FIG. 1B shows a tandem dye with an acceptor fluorophore and “n” donor fluorophores. A water solubilizing peptide links the acceptor fluorophore to the other sections of the compound, and the tandem dye is also bonded to a biomolecule.

FIG. 1C shows a tandem dye wherein a conjugated polymer donor fluorophore is linked to an acceptor fluorophore by a water solubilizing dye with one or more sulfonate groups. The tandem dye is also attached to a biomolecule.

FIG. 2 shows the chemical structure of an exemplary peptide with chemoselective groups, thereby allowing it to be selectively attached to two different groups.

FIG. 3 shows exemplary peptides with various combinations of cysteic acid groups, chemoselective groups, and in some cases polyethylene glycol groups.

FIG. 4 shows synthetic routes to two different compounds that each have a water solubilizing peptide, a fluorophore, and a chemoselective group.

FIG. 5 shows a synthetic scheme for generating a tandem dye wherein two donor fluorophores and a biomolecule are attached to a peptide scaffold, and a water solubilizing peptide with sulfonate groups links the scaffold to the acceptor fluorophore.

FIG. 6 shows further examples of water solubilizing peptides with sulfonate groups in various configurations.

FIG. 7 shows two water solubilizing peptides with either four or six sulfonate groups.

### DEFINITIONS

“Alkyl” refers to a monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases the alkyl group has 1 to 24 carbon atoms, e.g. 1 to 12, 1 to 6, or 1 to 3.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl.

“Alkynyl” refers to a monoradical, branched or linear, non-cyclic hydrocarbonyl group that comprises a carbon-carbon triple bond. Exemplary alkynyl groups include ethynyl and n-propynyl.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g., O, S, N) as a ring atom and that is not aromatic (i.e., distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e., none of the ring atoms are heteroatoms (e.g., O, S, N). In some cases, the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the aromatic ring is a heteroatom (e.g., O, S, N). Exemplary heteroaryl groups include those obtained from removing a hydrogen atom from pyridine, pyrimidine, furan, thiophene, or benzothiophene.

The term “substituted” refers the removal of one or more hydrogens from an atom (e.g., from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no long be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

Exemplary substituents include alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, sulfonate, and substituted versions thereof.

In some cases, the substitutions can themselves be further substituted with one or more groups. For example, the group —C₆H₄CH₂CH₃ can be considered as substituted aryl, i.e., an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein. In some cases, the substituents are not substituted with any other groups.

Diradical groups are also described herein, i.e., in contrast to the monoradical groups such as alkyl and aryl described above. The term “alkylene” refers to the diradical version of an alkyl group, i.e., an alkylene group is a diradical, branched or linear, cyclic or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e., a CHCH₃ fragment where the first atom has two single bonds to other two different groups). The term “arylene” refers to the diradical version of an aryl group, e.g., 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Acyl” refers to a group of formula —C(O)R wherein R is alkyl, alkenyl, alkynyl, or substituted versions thereof. For example, the acetyl group has formula —C(O)CH₃. “Carbonyl” refers to a diradical group of formula —C(O)—.

“Alkoxy” refers to a group of formula —O(alkyl). Similar groups can be derived from alkenyl, alkynyl, aryl, heteroaryl, and other groups.

“Amino” refers to the group —NR^{X}R^{Y} wherein R^{X} and R^{Y} are each independently H or a non-hydrogen substituent. Exemplary non-hydrogen substituents include alkyl groups (e.g., methyl, ethyl, and isopropyl).

“Carbonyl” refers to a diradical group of formula —C(O)—.

“Carboxy” is used interchangeably with carboxyl and carboxylate to refer to the —CO₂H group and salts thereof.

“Ether” refers to a diradical group of formula —O—. For instance, if the ether group is connected to an alkyl group, then the overall group is an alkoxy group (e.g. —OCH₃ or methoxy). If the ether is connected to a carbonyl group, then the overall group is an ester group of formula —OC(O)—.

“Halo” and “halogen” refer to the chloro, bromo, fluoro, and iodo groups.

“Nitro” refers to the group of formula —NO₂.

Unless otherwise specified, reference to an atom is meant to include all isotopes of that atom. For example, reference to H includes ¹H, ²H (i.e., D or deuterium) and ³H (i.e., tritium), and reference to C is includes both ¹²C and all other isotopes of carbon (e.g., ¹³C). Unless specified otherwise, groups include all possible stereoisomers.

The terms “reactive moiety”, “chemoselective functional group”, “chemoselective tag”, and “conjugation tag” are used interchangeably and refer to a functional group that can selectively react with another compatible functional group to form a covalent bond, in some cases, after optional activation of one of the functional groups. Chemoselective functional groups of interest include, but are not limited to, thiols and maleimide or iodoacetamide, amines and carboxylic acids or active esters thereof, as well as groups that can react with one another via Click chemistry, e.g., azide and alkyne groups (e.g., cyclooctyne groups), tetrazine, transcyclooctene, dienes and dienophiles, and azide, sulfur(VI) fluoride exchange chemistry (SuFEX), sulfonyl fluoride, as well as hydroxyl, hydrazido, hydrazino, aldehyde, ketone, azido, alkyne, phosphine, epoxide, succinimide and the like.

As used herein, the term “sample” relates to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g., body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant.

Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

The terms “support bound” and “linked to a support” are used interchangeably and refer to a moiety (e.g., a specific binding member) that is linked covalently or non-covalently to a support of interest. Covalent linking may involve the chemical reaction of two compatible functional groups (e.g., two chemoselective functional groups, an electrophile and a nucleophile, etc.) to form a covalent bond between the two moieties of interest (e.g., a support and a specific binding member). In some cases, non-covalent linking may involve specific binding between two moieties of interest (e.g., two affinity moieties such as a hapten and an antibody or a biotin moiety and a streptavidin, etc.). In certain cases, non-covalent linking may involve absorption to a substrate.

The terms “peptide” and “polypeptide” are used interchangeably herein to refer to a polymeric form of amino acids of any length, including peptides that range from 2 to 500 amino acids in length, such as from 2 to 350 amino acids, from 2 to 200 amino acids, from 2 to 100 amino acids, from 2 to 50 amino acids, and from 2 to 25 amino acids. In some cases, the peptide has 3 or more amino acids, such as 5 or more, 10 or more, or 20 or more. The terms peptide and polypeptide are also used interchangeably with the term “protein”. The amino acids in the peptide can be coded and non-coded amino acids, chemically or biochemically modified or derivatized amino acids, and peptides having a modified backbone in which the conventional backbone has been replaced with non-naturally occurring or synthetic backbones.

The terms “polyethylene oxide”, “PEO”, “polyethylene glycol” and “PEG” are used interchangeably and refer to a polymeric group including a chain described by the formula —(CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, acyloxy, and amido terminal and/or substituent groups. PEG groups are also described by S. Zalipsky in “Functionalized poly(ethylene glycol) for preparation of biologically relevant conjugates”, Bioconjugate Chemistry 1995, 6 (2), 150-165; by Zhu et al in “Water-Soluble Conjugated Polymers for Imaging, Diagnosis, and Therapy”, Chem. Rev., 2012, 112 (8), pp 4687-4735, “Poly(ethylene glycol) Chemistry: Biotechnical and Biomedical Applications”, J. M. Harris, Ed., Plenum Press, New York, N.Y. (1992); and “Poly(ethylene glycol) Chemistry and Biological Applications”, J. M. Harris and S. Zalipsky, Eds., ACS (1997); and International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964, and U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966).

As used herein the term “isolated,” refers to a moiety of interest that is at least 60% free, at least 75% free, at least 90% free, at least 95% free, at least 98% free, and even at least 99% free from other components with which the moiety is associated with prior to purification.

As used herein, the terms “evaluating”, “determining,” “measuring,” and “assessing,” and “assaying” are used interchangeably and include both quantitative and qualitative determinations.

The term “separating”, as used herein, refers to physical separation of two elements (e.g., by size or affinity, etc.) as well as degradation of one element, leaving the other intact.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some cases, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some cases, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3, or 4 atoms, of the cyclic group are included in the backbone. A linker may be cleavable or non-cleavable.

As used herein, the terms “water solubilizing group”, “water soluble group” and WSG are used interchangeably and refer to a group or substituent that is well solvated in aqueous environments e.g., under physiological conditions, and which imparts improved water solubility upon the molecule to which it is attached. A WSG can increase the solubility of a tandem dye or component thereof, e.g., donor or acceptor fluorophore, in a predominantly aqueous solution, as compared to a control tandem dye or component thereof which lacks the WSG. In some cases, the WSG can increase the solubility of a compound (e.g., a dye, a tandem dye, or a labeled specific binding member) as compared to a control compound wherein the WSG is replaced with a hydrogen atom. In some cases, the WSG increases the solubility in an aqueous medium, e.g., distilled water, by 1% or more, such as by 10% or more, 25% or more, 50% or more, 100% or more, or 500% or more. The water solubilizing groups may be any convenient hydrophilic group that is well solvated in aqueous environments.

The water soluble group (WSG) can be capable of imparting solubility in water in excess of 10 mg/mL to the subject dye or polymeric tandem dye, such as in excess of 20 mg/mL, in excess of 30 mg/mL, in excess of 40 mg/mL, in excess of 50 mg/mL, in excess of 60 mg/mL, in excess of 70 mg/mL, in excess of 80 mg/mL, in excess of 90 mg/mL or in excess of 100 mg/mL. In certain cases, the branched non-ionic water soluble group (WSG) is capable of imparting solubility in water (e.g., an aqueous buffer) of 20 mg/mL or more to the subject dye or polymeric tandem dye, such as 30 mg/mL or more, 40 mg/mL or more, 50 mg/mL or more, 60 mg/mL or more, 70 mg/mL or more, 80 mg/mL or more, 90 mg/mL or more, 100 mg/mL or more, or even more. It is understood that water-soluble dipyrromethene-based dye may, under certain conditions, form discrete water solvated nanoparticles in aqueous systems. In certain cases, the water solvated nanoparticles are resistant to aggregation and find use in a variety of biological assays.

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C.

The specific binding member can be proteinaceous. As used herein, the term “proteinaceous” refers to a moiety that is composed of amino acid residues. A proteinaceous moiety can be a polypeptide. In certain cases, the proteinaceous specific binding member is an antibody. In certain embodiments, the proteinaceous specific binding member is an antibody fragment, e.g., a binding fragment of an antibody that specifically binds to a a target analyte. As used herein, the terms “antibody” and “antibody molecule” are used interchangeably and refer to a protein consisting of one or more polypeptides substantially encoded by all or part of the recognized immunoglobulin genes.

The recognized immunoglobulin genes, for example in humans, include the kappa (k), lambda (l), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The term antibody is meant to include full length antibodies and may refer to a natural antibody from any organism, an engineered antibody, or an antibody generated recombinantly for experimental, therapeutic, or other purposes as further defined below.

Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions.

In certain embodiments, the specific binding member is a Fab fragment, a F(ab′)₂ fragment, a scFv, a diabody or a triabody. In certain embodiments, the specific binding member is an antibody. In some cases, the specific binding member is a murine antibody or binding fragment thereof. In certain instances, the specific binding member is a recombinant antibody or binding fragment thereof.”

“Active pharmaceutical ingredient” (API), “active agent”, “pharmacologically active agent”, and “drug” are used interchangeably herein to refer to a chemical material or compound which, when administered to an organism (e.g., a human or non-human animal) induces a desired pharmacologic and/or physiologic effect by local and/or systemic action.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

A “plurality” contains at least 2 members. In certain cases, a plurality may have 5 or more, such as 6 or more, 7 or more, 8 or more, 9 or more, 10 or more, 20 or more, 30 or more, 40 or more, 50 or more, 60 or more, 70 or more, 80 or more, 90 or more, 100 or more, 300 or more, 1000 or more, 3000 or more, 10,000 or more, 100,000 or more members.

Numeric ranges are inclusive of the numbers defining the range.

### DETAILED DESCRIPTION

Sulfonate-containing water-solubilizing peptides are provided. Such water-solubilizing peptides can be attached to, for example, a dye or a tandem dye to increase the solubility thereof. Also provided are methods of using the dyes that include the water-solubilizing peptides, as well as kits including such dyes.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Dyes

The present disclosure provides dyes that include a fluorophore and a peptide comprising one or more sulfonate groups. The peptide comprising the sulfonate group can increase the solubility of the dye in water, and hence the peptide can be referred to as a water-solubilizing peptide. The terms “fluorophore” and “chromophore” are used interchangeably herein.

As used herein, the term “sulfonate group” refers to a moiety of formula —SO₃H or a salt thereof. The form of the sulfonate group can change based on properties of a liquid in which the dye is dissolved or dispersed, e.g., the pH of an aqueous liquid. In some cases, the sulfonate group has the formula —SO₃H. In some embodiments, the sulfonate group is monovalent salt, e.g., —SO₃⁻ Li⁺, —SO₃⁻ Na⁺, —SO₃⁻ K⁺, —SO₃⁻ NH₄⁺, and —SO₃⁻ N(CH₃)₄⁺. In some cases, the sulfonate group is a divalent salt wherein the divalent cation is shared among different groups, e.g., wherein the divalent salt has the form —SO₃⁻ (½ Ca²⁺) or —SO₃⁻ (½ Mg²⁺). In some cases, the sulfonate salt has an inorganic cation and in other cases the sulfonate salt has an organic cation.

The length of the water-solubilizing peptide may vary, as desired. In some embodiments, the water-solubilizing peptide includes 2 to 500 amino acid residues, such as from 2 to 350, from 2 to 200, from 2 to 100, and from 2 to 50. In some cases, the peptide has 3 or more amino acid residues, such as 5 or more amino acid residues, 10 or more amino acid residues, or 20 or more amino acid residues.

In certain cases, the peptide includes a cysteic acid residue. The chemical structures of cysteic acid and cysteine are shown below. Cysteic acid can be conceptualized as a cysteine molecule wherein the thiol (—SH) group has been oxidized to a sulfonate (—SO₃H) group.

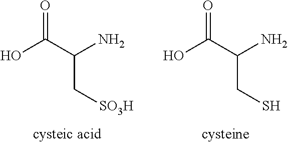 [SMILES: NC(CS(=O)(=O)O)C(=O)O ; NC(CS)C(=O)O]

For example, the peptide can include a cysteic acid group, as shown below. The peptide can also include a cysteic acid derivative group as illustrated below, wherein Y is a non-hydrogen substituent, e.g., alkyl, substituted alkyl, alkoxy, substituted alkoxy, or halo.

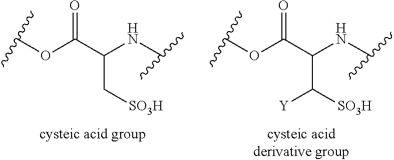

In some embodiments, the water-solubilizing peptide includes one or more non-cysteic acid residues. For example, the water-solubilizing peptide can include both a cysteic acid residue and a non-cysteic acid residue. In some cases, the peptide includes a sulfonate group that is part of a non-cysteic acid residue. In some embodiments, the peptide includes 5 or more non-cysteic acid residues, such as 10 or more non-cysteic acid residues, or 20 or more non-cysteic acid residues.

In some cases, the peptide has formula (I):

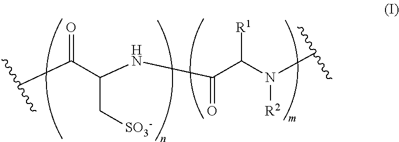

wherein:
each R¹ and R² is independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl, alkoxy, substituted alkoxy, amino, azido, carboxy, carboxamide, cyano, ether, halo, hydroxy, nitro, thiol, thioether, thioketo, guanidino, imidazole, indole, borate, —SO₂, and —SO₃—, provided that R¹ and R² can together with the atoms to which they are attached form a heterocyclic group; and
n is an integer ranging from 1 to 200 and m is an integer ranging from 0 to 200, provided that n+m is 2 or more.

As used herein, “n” and “m” refer to the number of monomeric units included in the peptide. These monomers can be ordered in any manner, such as a block copolymer (e.g., N—N—N-M-M-M), an alternating copolymer (e.g., N-M-N-M-N-M), or a random copolymer (e.g., N—N-M-N-M-M-M-N).

As stated above, n+m is 2 or more since the peptide includes multiple monomeric residues. For example, in some embodiments m is 0 and therefore n is an integer ranging from 2 to 200, and therefore the peptide is composed of cysteic acid residues. In other examples, m is an integer ranging from 1 to 200, and since n is an integer of 1 to 200 then the peptide is composed of a combination of cysteic acid residues and non-cysteic acid residues.

The “m” subunit of formula (I) can represent any naturally-occurring or non-naturally-occurring amino acid group. As such, if the “m” subunit represents a naturally-occurring amino acid, then the R¹ group will correspond to that amino acid, e.g., R¹ would be —CH₂CO₂H for aspartic acid, R¹ would be —CH₃ for alanine, and R¹ would be H for glycine. In some embodiments, each R¹ is independently H, alkyl, substituted alkyl, heterocycle, or substituted heterocycle. In some cases, R² is H.

In some cases, n is an integer ranging from 1 to 100, such as from 1 to 50, from 1 to 20, from 2 to 100, from 2 to 50, or from 2 to 20.

In some embodiments, m is 0 and therefore the peptide has formula (II):

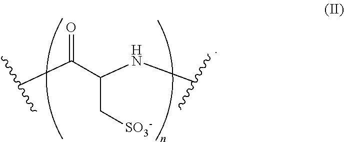

In some cases, the dye comprises an organic dye. Organic dyes may vary, wherein organic dyes that may be modified with the water-solubilizing peptides with sulfonate groups of the invention include, but are not limited to: cyanine dyes, rhodamine dyes, xanthene dyes, coumarin dyes, polymethines, pyrenes, dipyrromethene borondifluorides, napthalimides, thiazine dyes, and acridine dyes. Organic dyes of interest include, but are not limited to, fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY FL, BODIPY FL-Br₂, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, Dyonomics dyes (e.g. DY 431, DY 485XL, DY 500XL, DY 610, DY 640, DY 654, DY 682, DY 700, DY 701, DY 704, DY 730, DY 731, DY 732, DY 734, DY 752, DY 778, DY 782, DY 800, DY 831), dipyrromethene borondifluoride (BODIPY), Biotium CF 555, diethylamino coumarin, and derivatives thereof. In some cases, the water-solubilizing peptide is bound to the organic dye.

In some embodiments, the dye further comprises a non-conjugated polymeric backbone comprising non-conjugated repeat units. By “non-conjugated” is meant that at least a portion of the repeat unit includes a saturated backbone group (e.g., a group having two or more consecutive single covalent bonds) which precludes pi conjugation or an extended delocalized electronic structure along the polymeric backbone from one repeat unit to the next. It is understood that even though one repeat unit may not be conjugated to an adjacent repeat unit, such a repeat unit may include one or more isolated unsaturated groups including an unsaturated bond (e.g., of an alkenylene group or an alkynylene group) and/or an aryl or heteroaryl group, which groups can be a part of the backbone. In some cases, each repeat unit of the polymeric backbone includes one sidechain including a linked pendant group or a chemo-selective tag for linking to a pendant group.

In certain embodiments, the polymeric backbone is a linear polymer. In certain cases, the linear polymer is selected from a peptide, a peptoid, a hydrocarbon polymer, and a PEG polymer. In certain cases, the linear polymer is a peptide. In certain cases, the linear polymer is a peptoid. In certain cases, the polymer is a hydrocarbon polymer. In certain other cases, the non-conjugated polymer is a PEG polymer. Further details regarding non-conjugated polymeric backbones that may be employed in embodiments of the invention are found in PCT application serial no. PCT/US2019/024662 published as WO 2019/191482 and PCT application serial no. PCT/US2020/019510 published as WO 2020/222894; the disclosures of which applications are herein incorporated by reference.

In certain instances, non-conjugated polymeric backbone is a peptide of from 2 to 100 amino acids, such as 2 to 90, 2 to 80, 2 to 70, 2 to 60, 2 to 50, 2 to 40 or 2 to 30 amino acids. In some cases, the linear peptide backbone includes 2 or more amino acids, such as 5 or more, 10 or more, 15 or more, 20 or more, 25 or more, 30 or more, up to a maximum of 100 amino acids. In certain cases, the tandem dye includes a linear peptide backbone of from 5 to 30 amino acids, such as 5 to 25, 5 to 20, 5 to 15, or 5 to 10 amino acids.

The non-conjugated repeat units may have any convenient configuration, such as a linear, branched or dendrimer configuration. The polymeric backbone can be a linear polymer. The polymeric backbone can be branched. In some instances, the dye includes a plurality of pendant chromophore groups each independently linked to a non-conjugated repeat unit of the polymeric backbone. The configuration of pendant groups can be installed during or after synthesis of the non-conjugated polymeric backbone. The incorporation of pendant groups can be achieved with a random configuration, a block configuration, or in a sequence-specific manner via stepwise synthesis, depending on the particular method of synthesis utilized.

In some instances, the non-conjugated repeat units comprise a plurality of amino acid residues. In some instances, the dye comprises an organic dye bound to the non-conjugated polymeric backbone. The water-solubilizing peptide can be bound to the non-conjugated polymeric backbone and/or to the organic dye.

In some instances, the dye is a polymeric dye (e.g., a fluorescent polymeric dye). Fluorescent polymeric dyes that find use in the subject methods and systems are varied. In some instances of the method, the polymeric dye includes a conjugated polymer. Conjugated polymers (CPs) are characterized by a delocalized electronic structure which includes a backbone of alternating unsaturated bonds (e.g., double and/or triple bonds) and saturated (e.g., single bonds) bonds, where π-electrons can move from one bond to the other. As such, the conjugated backbone may impart an extended linear structure on the polymeric dye, with limited bond angles between repeat units of the polymer. For example, proteins and nucleic acids, although also polymeric, in some cases do not form extended-rod structures but rather fold into higher-order three-dimensional shapes. In addition, CPs may form “rigid-rod” polymer backbones and experience a limited twist (e.g., torsion) angle between monomer repeat units along the polymer backbone chain. In some instances, the polymeric dye includes a CP that has a rigid rod structure. The structural characteristics of the polymeric dyes can have an effect on the fluorescence properties of the molecules.

Any convenient polymeric dye may be utilized in the subject devices and methods. In some instances, a polymeric dye is a multichromophore that has a structure capable of harvesting light to amplify the fluorescent output of a fluorophore. In some instances, the polymeric dye is capable of harvesting light and efficiently converting it to emitted light at a longer wavelength. In some cases, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer), and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “excitation light”) is at a wavelength which is absorbed by the light harvesting multichromophore system than when the signaling chromophore is directly excited by the pump light.

The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via Forster energy transfer.

Polymeric dyes of interest include, but are not limited to, those dyes described by Gaylord et al. in U.S. Publication Nos. 20040142344, 20080293164, 20080064042, 20100136702, 20110256549, 20110257374, 20120028828, 20120252986, 20130190193, the disclosures of which are herein incorporated by reference in their entirety; and Gaylord et al., J. Am. Chem. Soc., 2001, 123 (26), pp 6417-6418; Feng et al., Chem. Soc. Rev., 2010, 39, 2411-2419; and Traina et al., J. Am. Chem. Soc., 2011, 133 (32), pp 12600-12607, the disclosures of which are herein incorporated by reference in their entirety.

In some embodiments, the polymeric dye includes a conjugated polymer including a plurality of first optically active units forming a conjugated system, having a first absorption wavelength (e.g., as described herein) at which the first optically active units absorbs light to form an excited state. The conjugated polymer (CP) may be polycationic, polyanionic and/or a charge-neutral conjugated polymer.

The polymeric dye may have any convenient length. In some cases, the particular number of monomeric repeat units or segments of the polymeric dye may fall within the range of 2 to 500,000, such as 2 to 100,000, 2 to 30,000, 2 to 10,000, 2 to 3,000 or 2 to 1,000 units or segments, or such as 100 to 100,000, 200 to 100,000, or 500 to 50,000 units or segments.

The polymeric dyes may be of any convenient molecular weight (MW). In some cases, the MW of the polymeric dye may be expressed as an average molecular weight. In some instances, the polymeric dye has an average molecular weight of from 500 to 500,000, such as from 1,000 to 100,000, from 2,000 to 100,000, from 10,000 to 100,000 or even an average molecular weight of from 50,000 to 100,000. In certain embodiments, the polymeric dye has an average molecular weight of 70,000.

In certain instances, the polymeric dye includes the following structure:

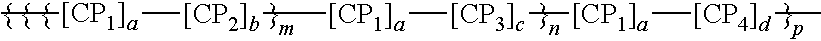

where CP₁, CP₂, CP₃ and CP₄ are independently a conjugated polymer segment or an oligomeric structure, wherein one or more of CP₁, CP₂, CP₃ and CP₄ are bandgap-lowering n-conjugated repeat units, and each n and each m are independently 0 or an integer from 1 to 10,000 and p is an integer from 1 to 100,000.

In some instances, the polymeric dye includes the following structure:

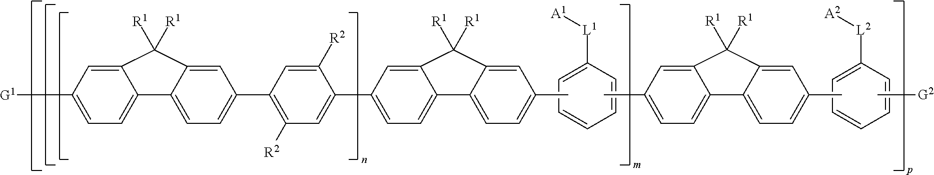

where each R¹ is independently a solubilizing group or a linker-dye; L¹ and L² are optional linkers; each R² is independently H or an aryl substituent; each A¹ and A² is independently H, an aryl substituent or a fluorophore; G¹ and G² are each independently selected from the group consisting of a terminal group, a π-conjugated segment, a linker and a linked specific binding member; each n and each m are independently 0 or an integer from 1 to 10,000; and p is an integer from 1 to 100,000. Solubilizing groups of interest include water-solubilizing peptide groups as described herein.

In some cases, the polymeric dye includes, as part of the polymeric backbone, a conjugated segment having one of the following structures:

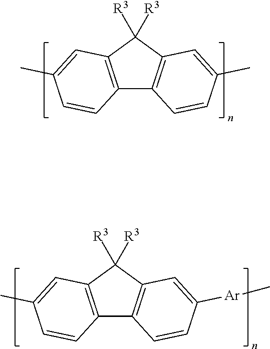

where each R³ is independently an optionally substituted alkyl or aryl group; Ar is an optionally substituted aryl or heteroaryl group; and each n is an integer from 1 to 10,000. In certain embodiments, R³ is an optionally substituted alkyl group. In certain embodiments, R³ is an optionally substituted aryl group. In some cases, R³ is substituted with a polyethyleneglycol, a dye, a chemoselective functional group or a specific binding moiety. In some cases, Ar is substituted with a polyethyleneglycol, a dye, a chemoselective functional group or a specific binding moiety.

In some instances, the polymeric dye includes the following structure:

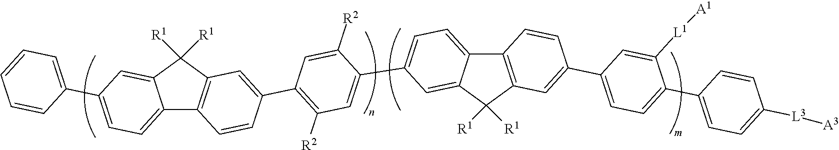

where each R¹ is independently a solubilizing group or a linker-dye group; each R² is independently H or an aryl substituent; each L¹ and L³ are independently optional linkers; each A¹ and A³ are independently H, a fluorophore, a functional group or a specific binding moiety (e.g., an antibody); and n and m are each independently 0 or an integer from 1 to 10,000, wherein n+m>1.

The polymeric dye may have one or more desirable spectroscopic properties, such as a particular absorption maximum wavelength, a particular emission maximum wavelength, extinction coefficient, quantum yield, and the like (see e.g., Chattopadhyay et al., “Brilliant violet fluorophores: A new class of ultrabright fluorescent compounds for immunofluorescence experiments.” Cytometry Part A, 81A(6), 456-466, 2012). In some embodiments, the polymeric dye has an absorption curve between 280 nm and 475 nm. In certain embodiments, the polymeric dye has an absorption maximum (excitation maximum) in the range 280 nm and 475 nm. In some embodiments, the polymeric dye absorbs incident light having a wavelength in the range between 280 nm and 475 nm. In some embodiments, the polymeric dye has an emission maximum wavelength ranging from 400 nm to 850 nm, such as 415 nm to 800 nm, where specific examples of emission maxima of interest include, but are not limited to: 421 nm, 510 nm, 570 nm, 602 nm, 650 nm, 711 nm and 786 nm. In some instances, the polymeric dye has an emission maximum wavelength in a range selected from the group consisting of 410 nm to 430 nm, 500 nm to 520 nm, 560 nm to 580 nm, 590 nm to 610 nm, 640 nm to 660 nm, 700 nm to 720 nm, and 775 nm to 795 nm. In certain embodiments, the polymeric dye has an emission maximum wavelength of 421 nm. In some instances, the polymeric dye has an emission maximum wavelength of 510 nm. In some cases, the polymeric dye has an emission maximum wavelength of 570 nm. In certain embodiments, the polymeric dye has an emission maximum wavelength of 602 nm. In some instances, the polymeric dye has an emission maximum wavelength of 650 nm. In certain cases, the polymeric dye has an emission maximum wavelength of 711 nm. In some embodiments, the polymeric dye has an emission maximum wavelength of 786 nm. In certain instances, the polymeric dye has an emission maximum wavelength of 421 nm±5 nm. In some embodiments, the polymeric dye has an emission maximum wavelength of 510 nm±5 nm. In certain instances, the polymeric dye has an emission maximum wavelength of 570 nm±5 nm. In some instances, the polymeric dye has an emission maximum wavelength of 602 nm±5 nm. In some embodiments, the polymeric dye has an emission maximum wavelength of 650 nm±5 nm. In certain instances, the polymeric dye has an emission maximum wavelength of 711 nm±5 nm. In some cases, the polymeric dye has an emission maximum wavelength of 786 nm±5 nm. In certain embodiments, the polymeric dye has an emission maximum selected from the group consisting of 421 nm, 510 nm, 570 nm, 602 nm, 650 nm, 711 nm and 786 nm.

In some instances, the polymeric dye has an extinction coefficient of 1×10⁶ cm⁻¹M⁻¹ or more, such as 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In certain embodiments, the polymeric dye has a quantum yield of 0.05 or more, such as 0.1 or more, 0.15 or more, 0.2 or more, 0.25 or more, 0.3 or more, 0.35 or more, 0.4 or more, 0.45 or more, 0.5 or more, or even more. In certain cases, the polymeric dye has a quantum yield of 0.1 or more. In certain cases, the polymeric dye has a quantum yield of 0.3 or more. In certain cases, the polymeric dye has a quantum yield of 0.5 or more. In some embodiments, the polymeric dye has an extinction coefficient of 1×10⁶ or more and a quantum yield of 0.3 or more. In some embodiments, the polymeric dye has an extinction coefficient of 2×10⁶ or more and a quantum yield of 0.5 or more.

Specific polymeric dyes that may be employed include, but are not limited to, BD Horizon Brilliant™ Dyes, such as BD Horizon Brilliant™ Violet Dyes (e.g., BV421, BV510, BV605, BV650, BV711, BV786); BD Horizon Brilliant™ Ultraviolet Dyes (e.g., BUV395, BUV496, BUV737, BUV805); and BD Horizon Brilliant™ Blue Dyes (e.g., BB515) (BD Biosciences, San Jose, CA).

In some cases, the water solubilizing peptide is a pendant group, i.e., it is located at an end of the dye. In such cases, the pendant water solubilizing peptide will be a monoradical. In other cases, the water solubilizing peptide is a linker group, i.e., it is located between two or more groups. For example, the linker water solubilizing peptide can be a diradical that links two different groups.

### Tandem Dyes

Also provided are tandem dyes that include a donor fluorophore, an acceptor fluorophore, and a water-solubilizing peptide comprising a sulfonate group, e.g., as described herein. The water-solubilizing peptide comprising the sulfonate group can increase the solubility of the dye in water, and hence the peptide can be referred to as a water-solubilizing peptide. Tandem dyes are compounds having two covalently linked different fluorophores, which fluorophores may be covalently linked to each other directly or through a linking group. One of the fluorophores serves as donor fluorophore and the other fluorophore acts as acceptor fluorophore. The donor and acceptor fluorophores together form a fluorescence-resonance energy transfer (FRET) pair. Such FRET pairs behave as a unique dye that has the excitation properties of the donor fluorophore and the emission properties of the acceptor fluorophore.

Excitation of the donor can lead to energy transfer to, and emission from, the covalently attached acceptor fluorophore. Mechanisms for energy transfer between the donor chromophores to a linked acceptor signaling fluorophore include, for example, resonant energy transfer (e.g., Forster (or fluorescence) resonance energy transfer, FRET), quantum charge exchange (Dexter energy transfer) and the like. These energy transfer mechanisms can be relatively short range; that is, close proximity of chromophores of the light harvesting multichromophore system to each other and/or to an acceptor fluorophore provides for efficient energy transfer. Under conditions for efficient energy transfer, amplification of the emission from the acceptor fluorophore can occur where the emission from the luminescent acceptor fluorophore is more intense when the incident light (the “pump light”) is at a wavelength which is absorbed by, and transferred from, the chromophores of the light harvesting chromophore than when the luminescent acceptor fluorophore is directly excited by the pump light. By “efficient” energy transfer is meant 10% or more, such as 20% or more or 30% or more, 40% or more, 50% or more, of the energy harvested by the donor chromophores is transferred to the acceptor. By “amplification” is meant that the signal from the acceptor fluorophore is 1.5× or greater when excited by energy transfer from the donor light harvesting chromophore system as compared to direct excitation of the acceptor fluorophore with incident light of an equivalent intensity. The signal may be measured using any convenient method. In some cases, the 1.5× or greater signal refers to an intensity of emitted light. In certain cases, the 1.5× or greater signal refers to an increased signal to noise ratio. In certain embodiments of the tandem dye, the acceptor fluorophore emission is 1.5 fold greater or more when excited by the chromophore as compared to direct excitation of the acceptor fluorophore with incident light, such as 2-fold or greater, 3-fold or greater, 4-fold or greater, 5-fold or greater, 6-fold or greater, 8-fold or greater, 10-fold or greater, 20-fold or greater, 50-fold or greater, 100-fold or greater, or even greater as compared to direct excitation of the acceptor fluorophore with incident light.

In some instances, the tandem dye exhibits an effective Stokes shift ranging from 25 nm to 300 nm, such as from 50 nm to 250 nm or from 75 nm to 200 nm. In some cases the effective Stokes shift is 25 nm or more, such as 50 nm or more, 75 nm or more, 100 nm or more, such as 110 nm or more, 120 nm or more, 130 nm or more, 140 nm or more, 150 nm or more, 160 nm or more, 170 nm or more, 180 nm or more, 190 nm or more, 200 nm or more, 250 nm or more when the light harvesting chromophore is directly excited with incident light.

The emission of the tandem dye can have a quantum yield of 0.03 or more, such as a quantum yield of 0.04 or more, 0.05 or more, 0.06 or more, 0.07 or more, 0.08 or more, 0.09 or more, 0.1 or more, 0.15 or more, 0.2 or more, 0.3 or more or even more. In some instances, the polymeric tandem dye has an extinction coefficient of 5×10⁵ cm⁻¹M⁻¹ or more, such as 6×10⁵ cm⁻¹M⁻¹ or more, 7×10⁵ cm⁻¹M⁻¹ or more, 8×10⁵ cm⁻¹M⁻¹ or more, 9×10⁵ cm⁻¹M⁻¹ or more, such as 1×10⁶ cm⁻¹M⁻¹ or more, 1.5×10⁶ cm⁻¹M⁻¹ or more, 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In some embodiments, the tandem dye has a molar extinction coefficient of 5×10⁵ M⁻¹cm⁻¹ or more. In certain embodiments, the tandem dye has a molar extinction coefficient of 1×10⁶ M⁻¹cm⁻¹ or more.

At least one, and in some cases both, of the donor fluorophore and the acceptor fluorophore comprise an organic dye. For instance, the organic dye of the donor fluorophore can be selected from the group consisting of cyanine dyes, rhodamine dyes, xanthene dyes, coumarin dyes, polymethines, pyrenes, dipyrromethene borondifluorides, napthalimides, thiazine dyes, and acridine dyes. In some cases, the organic dye of the acceptor fluorophore can be selected from the group consisting of cyanine dyes, rhodamine dyes, xanthene dyes, coumarin dyes, polymethines, pyrenes, dipyrromethene borondifluorides, napthalimides, thiazine dyes, and acridine dyes.

Organic dyes of interest that may be employed as donors or acceptors include, but are not limited to, fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY FL, BODIPY FL-Br.sub.2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, Dyonomics dyes (e.g. DY 431, DY 485XL, DY 500XL, DY 610, DY 640, DY 654, DY 682, DY 700, DY 701, DY 704, DY 730, DY 731, DY 732, DY 734, DY 752, DY 778, DY 782, DY 800, DY 831), dipyrromethene borondifluoride (BODIPY), Biotium CF 555, diethylamino coumarin, and derivatives thereof.

As discussed above, a dye can include a non-conjugated polymeric backbone. In a similar manner, in some embodiments the tandem dye can comprise a non-conjugated polymeric backbone comprising non-conjugated repeat units. In some embodiments the non-conjugated repeat units include a plurality of amino acid residues. The water-solubilizing peptide can be bound to the donor fluorophore, the acceptor fluorophore, and/or the non-conjugated polymeric backbone.

As described above, the dyes can include a fluorophore that is a conjugated polymer. As such, in some cases the donor fluorophore of the tandem dye can comprise a conjugated polymer. As described above in relation to dyes, in some cases the conjugated polymer can include a series of optionally-substituted aryl and/or heteroaryl groups. Aryl groups of interest include fluorene and phenyl whereas heteroaryl groups of interest include thiophene, pyridine, and BODIPY groups. In such cases, the water-solubilizing peptide can be bound to the conjugated polymer and/or the acceptor fluorophore.

In some cases, the water solubilizing peptide is a pendant group, i.e., it is located at an end of the dye. In such cases, the pendant water solubilizing peptide will be a monoradical. In other cases, the water solubilizing peptide is a linker group, i.e., it is located between two or more groups. For example, the linker water solubilizing peptide can be a diradical that links two different groups, e.g., an organic dye and a specific binding member.

In some cases, the water solubilizing peptide is a pendant group, i.e., it is located at an end of the tandem dye. In such cases, the pendant water solubilizing peptide will be a monoradical. In other cases, the water solubilizing peptide is a linker group, i.e., it is located between two or more groups. For example, the linker water solubilizing peptide can be a diradical that links two different groups. For instance, the water solubilizing peptide can be bonded to the acceptor chromophore and to a non-conjugated backbone, or the water solubilizing peptide is bonded to a donor chromophore and to a non-conjugated backbone.

### Labeled Specific Binding Members

Aspects of the present disclosure also include labeled specific binding members that include a dye or tandem dye, e.g., as described above, along with a specific binding member. Stated in another manner, the specific binding member is labeled with the dye or tandem dye, e.g., to allow for detection of a target analyte that binds to the specific binding member.

As discussed above, the water solubilizing peptide can be a pendant group. As such, the peptide can be a terminal, monoradical group bonded to a fluorophore or a specific binding member. In other cases, the peptide can be a linker that connects two or more groups. For instance, the peptide can connect an acceptor chromophore to the remainder of a tandem dye, e.g., wherein the water solubilizing peptide is bonded to the acceptor chromophore and to a non-conjugated backbone. In other instances, the water solubilizing peptide is bonded to a donor chromophore and a non-conjugated backbone, or the water solubilizing peptide is bonded to a specific binding member and a non-conjugated backbone.

Methods of Increasing the Solubility of a Compound with a Peptide Comprising A Sulfonate Group

The present disclosure provides methods of increasing the solubility of a compound with a peptide comprising a sulfonate group, as compared to the solubility of the compound without the peptide comprising a sulfonate group component. As such, the peptide comprising a sulfonate group can be considered a water solubilizing group. In some instances the method includes the step of bonding the water solubilizing peptide to the compound. The water solubilizing peptide can be bonded covalently or non-covalently (e.g., ionically) to the compound.

In some embodiments, the compound being solubilized by the water solubilizing peptide is a dye, e.g., as described above, which includes a fluorophore. In other cases, the compound being solubilized is a tandem dye including a donor fluorophore and an acceptor fluorophore, e.g., as discussed above. In some cases, the compound is a specific binding member, e.g., that has been labeled with a dye or a tandem dye.

In some cases, the compound being solubilized is an active pharmaceutical ingredient (API). In other cases, the compound is selected from the group consisting of a pharmaceutically acceptable excipient, a pharmaceutically acceptable diluent, a pharmaceutically acceptable carrier, and a pharmaceutically acceptable adjuvant.

For instance, some known compounds have attached polyethylene glycol (PEG) groups, and are thus referred to as “PEGylated” compounds. water-solubilizing peptide groups can be bonded instead of, or in addition to, the PEG groups using the presently described methods. For example, Harris et al. describes polypeptide drugs with attached PEG groups (Nature Reviews Drug Discovery, 2003, 2, 214, doi:10.1038/nrd1033), and hence a polypeptide drug can be bonded to water-solubilizing peptide comprising the sulfonate group instead of such PEG groups according to methods of the present disclosure. Other compounds that can be functionalized with the water-solubilizing peptide, instead of or in addition to PEG, include dyes (e.g., Wu et al. (doi:10.1016/j.ejmech.2018.10.046) and Collado et al. (doi: 10.1039/C3RA46235H)), cytokines and therapeutic proteins (Francis et al., doi: 10.1016/s0925-5710(98)00039-5), small molecule agents (Li et al., 10.1016/j.progpolymsci.2012.07.006), excipients (e.g., PEGylated excipient in the COVID-19 vaccine from Pfizer, as discussed by Cabanillas et al. (doi: 10.1111/all.14711)), liposomes (Heger et al., doi: 10.1016/j.mvr.2009.02.006). In such cases, the compound (e.g., dye, cytokine, therapeutic protein, small molecule agent, excipient, or liposome) can be bonded to a water-solubilizing peptide to increase the solubility of the compound.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

### Methods of Labeling a Target Molecule

Also provided are methods of labeling a target molecule. Such methods include contacting the target molecule with a dye or tandem dye as described herein to covalently bond the target molecule to a reactive moiety of the dye or tandem dye, thereby producing the labelled target molecule.

Methods of interest for labelling a target, include but are not limited to, those methods and reagents described by Hermanson, Bioconjugate Techniques, Third edition, Academic Press, 2013. The contacting step may be performed in an aqueous solution. In some instances, the reactive moiety includes an amino functional group and the target molecule includes an activated ester functional group, such as a NHS ester or sulfo-NHS ester, or vice versa. In certain instances, the reactive moiety includes a maleimide functional group and the target molecule includes a thiol functional group, or vice versa. In certain instances, the reactive moiety includes an alkyne (e.g., a cyclooctyne group) functional group and the target molecule includes an azide functional group, or vice versa, which can be conjugated via Click chemistry.

Any convenient target molecules may be selected for labelling utilizing the subject methods. Target molecules of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell, a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially. In some embodiments, the target molecule is a specific binding member (e.g., as described herein). In certain instances, the specific binding member is an antibody. In some instances, the specific binding member is an antibody fragment or binding derivative thereof. In some case, the antibody fragment or binding derivative thereof is selected from the group consisting of a Fab fragment, a F(ab′)₂ fragment, a scFv, a diabody and a triabody.

In some cases, the method includes a separating step where the labelled target molecule is separated from the reaction mixture, e.g., excess reagents or unlabeled target. A variety of methods may be utilized to separate a target from a sample, e.g., via immobilization on a support, precipitation, chromatography, and the like.

In some instances, the method further includes detecting and/or analyzing the labelled target molecule. In some instances, the method further includes fluorescently detecting the labelled target molecule. Any convenient methods may be utilized to detect and/or analyze the labelled target molecule in conjunction with the subject methods and compositions. Methods of analyzing a target of interest that find use in the subject methods, include but are not limited to, flow cytometry, fluorescence microscopy, in-situ hybridization, enzyme-linked immunosorbent assays (ELISAs), western blot analysis, magnetic cell separation assays and fluorochrome purification chromatography. Detection methods of interest include but are not limited to fluorescence spectroscopy, fluorescence microscopy, nucleic acid sequencing, fluorescence in-situ hybridization (FISH), protein mass spectroscopy, flow cytometry, and the like.

Detection may be achieved directly via the polymeric tandem dye, or indirectly by a secondary detection system. The latter may be based on any one or a combination of several different principles including, but not limited to, antibody labelled anti-species antibody and other forms of immunological or non-immunological bridging and signal amplification systems (e.g., biotin-streptavidin technology, protein-A and protein-G mediated technology, or nucleic acid probe/anti-nucleic acid probes, and the like). Suitable reporter molecules may be those known in the field of immunocytochemistry, molecular biology, light, fluorescence, and electron microscopy, cell immunophenotyping, cell sorting, flow cytometry, cell visualization, detection, enumeration, and/or signal output quantification. More than one antibody of specific and/or non-specific nature might be labelled and used simultaneously or sequentially to enhance target detection, identification, and/or analysis.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

### Methods of Evaluating a Sample for Presence of a Target Analyte

Provided are methods of evaluating a sample for the presence of a target analyte by using a labeled specific binding member that includes a water-solubilizing peptide comprising a sulfonate group. In some embodiments the method includes:
(a) contacting the sample with a labeled specific binding member that specifically binds the target analyte to produce a labeled sample; and
(b) assaying the labeled composition for the presence of a labelled specific binding member-target analyte binding complex to evaluate whether the target analyte is present in the sample.

The labeled specific binding member employed in embodiments of methods of the invention includes a specific binding member conjugated to a dye or tandem dye, as described above. In the following section, the target analyte may be a target molecule of interest or reagent, e.g., primary antibody, bound to the target molecule, depending on whether the labeled specific binding member is employed as a primary or secondary label. Any convenient method may be used to contact the sample with a labeled specific binding member that specifically binds to the target analyte to produce the assay composition. In some instances, the sample is contacted with the labeled specific binding member under conditions in which the labeled specific binding member specifically binds to the target analyte, if present. For specific binding of the labeled specific binding member with the target analyte, an appropriate medium may be used that maintains the biological activity of the components of the sample and the signal domain antibody. The medium may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. Various media are commercially available and may be used according to the nature of the target analyte, including dMEM, HBSS, dPBS, RPMI, Iscove's medium, etc., in some cases supplemented with fetal calf serum or human platelet lysate. The final components of the medium, which may be a solution, may be selected depending on the components of the sample which are included. The temperature at which specific binding of the labeled specific binding member to the target analyte takes place may vary, and in some instances may range from 5° C. to 50° C., such as from 10° C. to 40° C., 15° C. to 40° C., 20° C. to 40° C., e.g., 20° C., 25° C., 30° C., 35° C. or 37° C. (e.g., as described above). In some instances, the temperature at which specific binding takes place is selected to be compatible with the biological activity of the specific binding member and/or the target analyte. In certain instances, the temperature is 25° C., 30° C., 35° C., or 37° C. In certain cases, the temperature at which specific binding takes place is room temperature (e.g., 25° C.), 30° C., 35° C., or 37° C. Any convenient incubation time for specific binding may be selected to allow for the formation of a desirable amount of binding complex, and in some instances, may be 1 minute (min) or more, such as 2 min or more, 10 min or more, 30 min or more, 1 hour or more, 2 hours or more, or even 6 hours or more.

Any convenient specific binding members may be utilized in the labeled specific binding members employed in methods of the invention. Specific binding members of interest include, but are not limited to, those specific binding members that specifically bind cell surface proteins of a variety of cell types, including but not limited to, stem cells, e.g., pluripotent stem cells, hematopoietic stem cells, T cells, T regulator cells, dendritic cells, B Cells, e.g., memory B cells, antigen specific B cells, granulocytes, leukemia cells, lymphoma cells, virus cells (e.g., HIV cells) NK cells, macrophages, monocytes, fibroblasts, epithelial cells, endothelial cells, and erythroid cells. Target cells of interest include cells that have a convenient cell surface marker or antigen that may be captured by a convenient specific binding member conjugate. In some embodiments, the target cell is selected from HIV containing cell, a Treg cell, an antigen-specific T-cell populations, tumor cells or hematopoetic progenitor cells (CD34+) from whole blood, bone marrow or cord blood. Any convenient cell surface proteins or cell markers may be targeted for specific binding to the conjugates employed in the subject methods. In some embodiments, the target cell includes a cell surface marker selected from a cell receptor and a cell surface antigen. In some cases, the target cell may include a cell surface antigen such as CD11b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71.

Any convenient targets may be selected for evaluation utilizing the subject methods. Targets of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell, a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, transcription factor, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially. In some embodiments, the polymeric dye conjugates include an antibody or antibody fragment. Any convenient target analyte that specifically binds an antibody or antibody fragment of interest may be targeted in the subject methods.

In some embodiments, the target analyte is associated with a cell. In certain instances, the target analyte is a cell surface marker of the cell. In certain cases, the cell surface marker is selected from the group consisting of a cell receptor and a cell surface antigen. In some instances, the target analyte is an intracellular target, and the method further includes treating the cell so as to provide access of the labeled specific binding member to the intracellular target, e.g., by permeabilizing or lysing the cell. As such, a labeled specific binding member employed in methods of the invention may target a cell surface or intracellular antigen. Alternatively, a labeled specific binding member employed in methods of the invention may target a primary antibody that in turn specifically binds to a target cell surface or intracellular antigen.

In some embodiments, the sample may include a heterogeneous cell population from which target cells are isolated. In some instances, the sample includes peripheral whole blood, peripheral whole blood in which erythrocytes have been lysed prior to cell isolation, cord blood, bone marrow, density gradient-purified peripheral blood mononuclear cells or homogenized tissue. In some cases, the sample includes hematopoetic progenitor cells (e.g., CD34+ cells) in whole blood, bone marrow or cord blood. In certain embodiments, the sample includes tumor cells in peripheral blood. In certain instances, the sample is a sample including (or suspected of including) viral cells (e.g., HIV).

The labeled specific binding members find use in the subject methods, e.g., for labeling a target cell, particle, target or analyte with a polymeric tandem fluorescent dye. For example, labeled specific binding members find use in labeling cells to be processed (e.g., detected, analyzed, and/or sorted) in a flow cytometer. The labeled specific binding members may include specific binding members, e.g., antibodies or binding fragments thereof, that specifically bind to, e.g., cell surface proteins of a variety of cell types (e.g., as described herein). The labeled specific binding members may be used to investigate a variety of biological (e.g., cellular) properties or processes such as cell cycle, cell proliferation, cell differentiation, DNA repair, T cell signaling, apoptosis, cell surface protein expression and/or presentation, and so forth. Labelled specific binding members may be used in any application that includes (or may include) antibody-mediated labeling of a cell, particle or analyte.

Aspects of the methods include assaying the assay composition, i.e., labeled specific binding member contacted sample, for the presence of a labeled specific binding member-target analyte binding complex to evaluate whether the target analyte is present in the sample. Once the sample has been contacted with the labeled specific binding member, any convenient method may be utilized in assaying the assay composition that is produced for the presence of a labeled specific binding member-target analyte binding complex. The labeled specific binding member-target analyte binding complex is the binding complex that is produced upon specific binding of the labeled specific binding member to the target analyte (or primary binding member, e.g., primary antibody, to the target antigen depending on the embodiment), if present. Assaying the assay composition may include detecting a fluorescent signal from the binding complex, if present. In some cases, the assaying includes a separating step where the target analyte, if present, is separated from the sample. A variety of methods can be utilized to separate a target analyte from a sample, e.g., via immobilization on a support. Assay methods of interest include, but are not limited to, any convenient methods and assay formats where pairs of specific binding members such as avidin-biotin or hapten-anti-hapten antibodies find use, are of interest. Methods and assay formats of interest that may be adapted for use with the subject compositions include, but are not limited to, flow cytometry methods, in-situ hybridization methods, enzyme-linked immunosorbent assays (ELISAs), western blot analysis, magnetic cell separation assays and fluorochrome purification chromatography.

In certain embodiments, the method further includes contacting the sample with a second specific binding member that specifically binds the target analyte. In certain instances, the second specific binding member is support bound. Any convenient supports may be utilized to immobilize a component of the subject methods (e.g., a second specific binding member). In certain instances, the support is a particle, such as a magnetic particle. In some instances, the second specific binding member and the polymeric dye conjugate produce a sandwich complex that may be isolated and detected, if present, using any convenient methods. In some embodiments, the method further includes flow cytometrically analyzing the polymeric dye conjugate-target analyte binding complex, i.e., a fluorescently labelled target analyte. Assaying for the presence of a labeled specific binding member-target analyte binding complex may provide assay results (e.g., qualitative or quantitative assay data) which can be used to evaluate whether the target analyte is present in the sample.

Any convenient supports may be utilized in the subject methods to immobilize any convenient component of the methods, e.g., labelled specific binding member, target, secondary specific binding member, etc. Supports of interest include, but are not limited to: solid substrates, where the substrate can have a variety of configurations, e.g., a sheet, bead, or other structure, such as a plate with wells; beads, polymers, particle, a fibrous mesh, hydrogels, porous matrix, a pin, a microarray surface, a chromatography support, and the like. In some instances, the support is selected from the group consisting of a particle, a planar solid substrate, a fibrous mesh, a hydrogel, a porous matrix, a pin, a microarray surface and a chromatography support. The support may be incorporated into a system that it provides for cell isolation assisted by any convenient methods, such as a manually-operated syringe, a centrifuge or an automated liquid handling system. In some cases, the support finds use in an automated liquid handling system for the high throughput isolation of cells, such as a flow cytometer.

In some embodiments of the method, the separating step includes applying an external magnetic field to immobilize a magnetic particle. Any convenient magnet may be used as a source of the external magnetic field (e.g., magnetic field gradient). In some cases, the external magnetic field is generated by a magnetic source, e.g. by a permanent magnet or electromagnet. In some cases, immobilizing the magnetic particles means the magnetic particles accumulate near the surface closest to the magnetic field gradient source, i.e. the magnet.

The separating may further include one or more optional washing steps to remove unbound material of the sample from the support. Any convenient washing methods may be used, e.g., washing the immobilized support with a biocompatible buffer which preserves the specific binding interaction of the polymeric dye and the specific binding member. Separation and optional washing of unbound material of the sample from the support provides for an enriched population of target cells where undesired cells and material may be removed.

In certain embodiments, the method includes detecting the labeled target analyte. Detecting the labeled target analyte may include exciting the polymeric fluorescent tandem dye with one or more lasers and subsequently detecting fluorescence emission from the polymeric fluorescent tandem dye using one or more optical detectors. Detection of the labeled target can be performed using any convenient instruments and methods, including but not limited to, flow cytometry, FACS systems, fluorescence microscopy; fluorescence, luminescence, ultraviolet, and/or visible light detection using a plate reader; high performance liquid chromatography (HPLC); and mass spectrometry. When using fluorescently labeled components in the methods and compositions of the present disclosure, it is recognized that different types of fluorescence detection systems can be used to practice the subject methods. In some cases, high throughput screening can be performed, e.g., systems that use 96 well or greater microtiter plates. A variety of methods of performing assays on fluorescent materials can be utilized, such as those methods described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In some cases, excitation radiation, from an excitation source having a first wavelength, passes through excitation optics. The excitation optics cause the excitation radiation to excite the sample. In response, fluorescently labelled targets in the sample emit radiation which has a wavelength that is different from the excitation wavelength. Collection optics then collect the emission from the sample. The device can include a temperature controller to maintain the sample at a specific temperature while it is being scanned. In certain instances, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

In some embodiments, the method of evaluating a sample for the presence of a target analyte further includes detecting fluorescence in a flow cytometer. In some embodiments, the method of evaluating a sample for the presence of a target analyte further includes imaging the labelling composition contacted sample using fluorescence microscopy. Fluorescence microscopy imaging can be used to identify a polymeric dye conjugate-target analyte binding complex in the contacted sample to evaluate whether the target analyte is present. Microscopy methods of interest that find use in the subject methods include laser scanning confocal microscopy.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

### Kits

Aspects of the invention further include kits for use in practicing the subject methods. The dyes, tandem dyes, labeled specific binding members, or a combination thereof can be included as reagents in kits either as starting materials or provided for use in, for example, the methodologies described above. Such dyes, tandem dyes, and labeled specific binging members can be provided with a container. Any convenient containers can be utilized, such as tubes, bottles, or wells in a multi-well strip or plate, a box, a bag, an insulated container, and the like. The subject kits can further include one or more components selected from a primer specific binding member for a given target analyte, a support bound specific binding member, a cell, a support, a biocompatible aqueous elution buffer, a control (positive and/or negative), etc., and instructions for use, as desired. A given kit may include reagents suitable for detection of a single target analyte, or multiple reagents suitable for detection of two or more different target analytes, e.g., where a given kit is configured for multiplex detection applications.

In certain embodiments, the kit finds use in evaluating a sample for the presence of a target analyte, such as an intracellular target. As such, in some instances, the kit includes one or more components suitable for permeabilizing or lysing cells. The one or more additional components of the kit may be provided in separate containers (e.g., separate tubes, bottles, or wells in a multi-well strip or plate).

In certain aspects, the kit further includes reagents for performing a flow cytometric assay. Reagents of interest include, but are not limited to, buffers for reconstitution and dilution, buffers for contacting a cell sample the chromophore, wash buffers, control cells, control beads, fluorescent beads for flow cytometer calibration and combinations thereof. The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The compositions of the kit may be provided in a liquid composition, such as any suitable buffer. Alternatively, the compositions of the kit may be provided in a dry composition (e.g., may be lyophilized), and the kit may optionally include one or more buffers for reconstituting the dry composition. In certain aspects, the kit may include aliquots of the compositions provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1: Arrangements with Water-Solubilizing Peptides

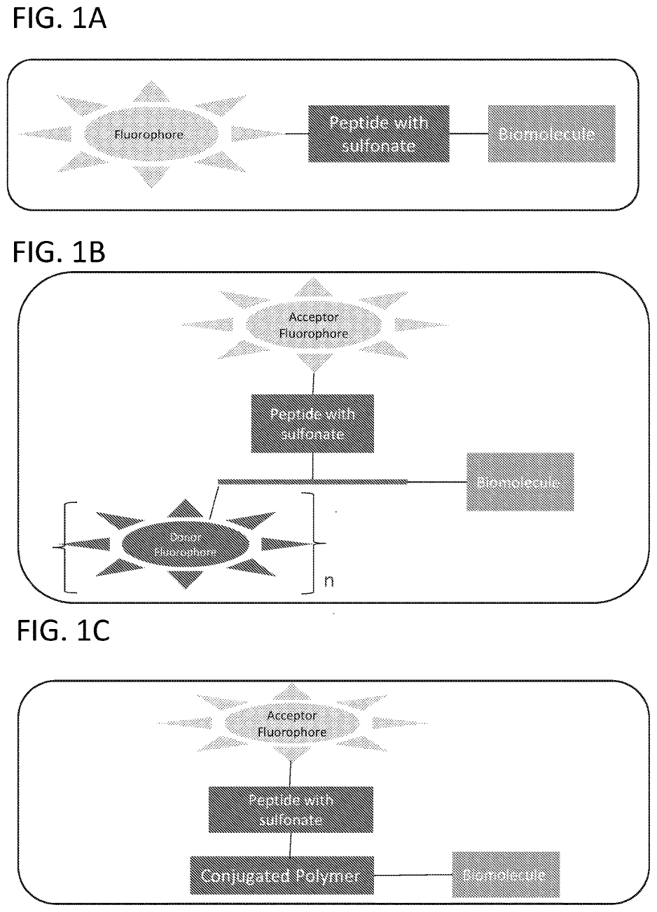

Compounds with water-solubilizing peptides are produced according to the embodiments of FIGS. 1A-C. In particular, FIG. 1A shows a compound including a fluorophore, a water-solubilizing peptide with a sulfonate group, and a biomolecule. The water-solubilizing peptide functions as a linker between the fluorophore and the biomolecule. The biomolecule can be a specific binding member, in which case the compound is a labeled specific binding member with increased water solubility due to the water solubilizing peptide.

FIG. 1B shows a corresponding embodiment with “n” donor fluorophores and an acceptor chromophore. For instance, n can be 1, or 2 or more. The water solubilizing peptide with the sulfonate group links the acceptor chromophore to the other sections of the compound. The compound will be a labeled specific binding member if the biomolecule is a specific binding member.

FIG. 1C shows a tandem dye that also includes a water-solubilizing peptide and a biomolecule. The conjugated polymer can function as a donor fluorophore that transfers energy to the acceptor fluorophore, and the water solubilizing peptide both links the acceptor chromophore to the other sections of the compound while also increasing water solubility.

### Example 2: Water Solubilizing Peptides Before and After Conjugation

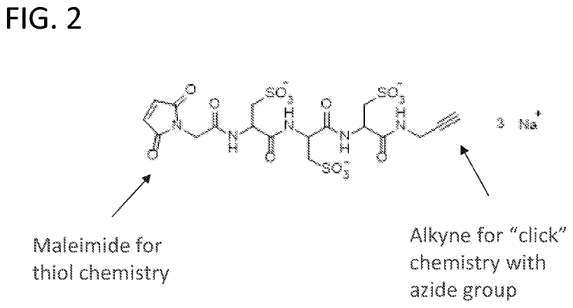

The precursor to the water solubilizing peptide can contain two different functional groups with orthogonal chemical reactivities. As such, the peptide can be covalently bonded to two different elements of the desired compound. FIG. 2 shows an embodiment of a water solubilizing peptide with three cysteic acid groups, and hence three sulfonate groups. The compound also includes one maleimide group that can chemoselectively react with a thiol group along with one alkyne group that can selectively react with an azide group. As such, the FIG. 2 embodiment can be covalently bonded to corresponding maleimide and azide groups. For instance, the FIG. 1A embodiment can be generated by reacting the maleimide of FIG. 2 with a thiol group on a fluorophore, and by reacting the alkyne group of FIG. 2 with an azide group on a biomolecule.

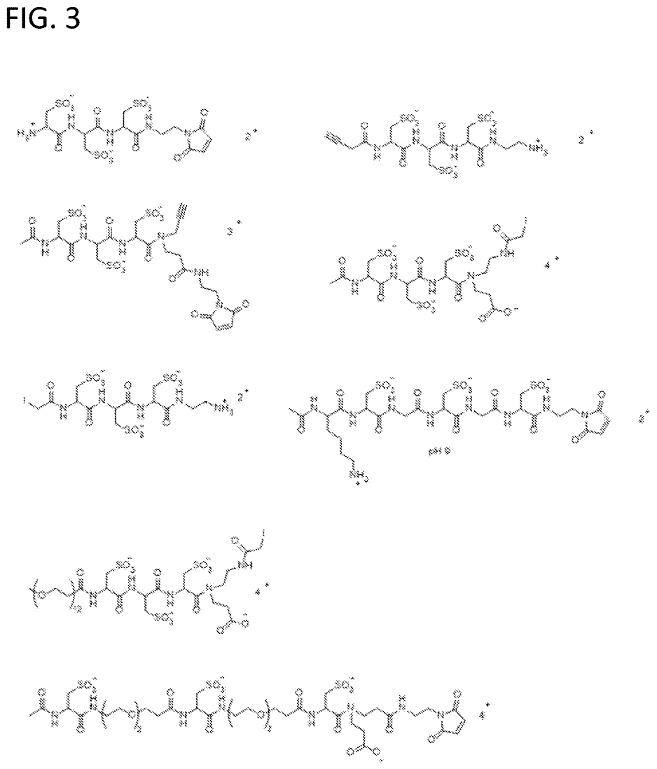

FIG. 3 shows additional examples of water solubilizing peptides that have various reactive moieties and arrangements of groups. In a similar manner to FIG. 2, some FIG. 3 embodiments have two different chemoselective functional groups, such as alkynes and maleimides, allowing the independent formation of covalent bonds to different group, thereby making the peptide into a linker. However, other FIG. 3 embodiments lack either a maleimide or alkyne, and such embodiments can be used simply as a terminal group that imparts increased water solubility. Also shown are terminal iodo groups, which can serve as electrophilic leaving groups, thereby allowing for covalent bond formation. Some FIG. 3 embodiments have terminal amino groups, which could be used as nucleophiles in covalent bond forming reactions. Furthermore, two of the FIG. 3 embodiments include polyethylene glycol units, which can be used in combination with the sulfonate groups to increase water solubility.

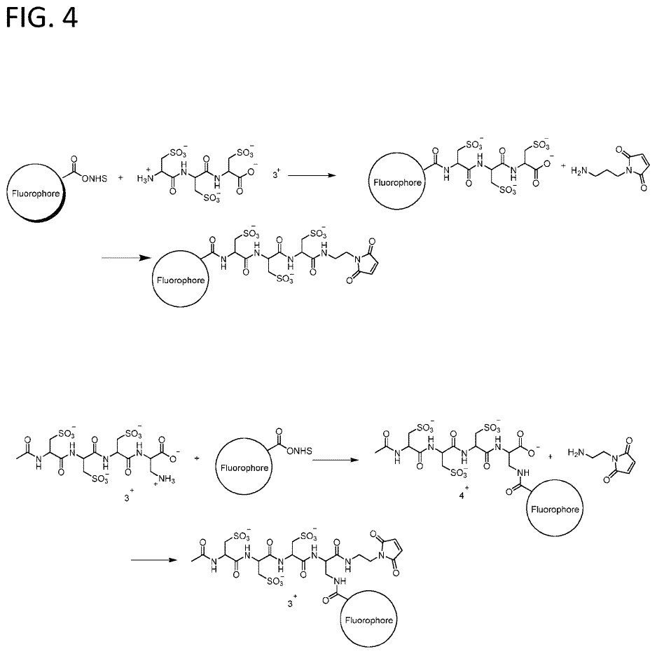

FIG. 4 provides exemplary synthetic routes whereby peptide groups are bonded to a fluorophores and chemoselective maleimide groups. In the top embodiment, a N-hydroxysuccinimide (NHS) group is reacted with an amino group to bond the fluorophore to the peptide. A maleimide group is also attached to the peptide-fluorophore group. Wherein the top embodiment the fluorophore is attached at and end of the peptide, in the bottom embodiment the fluorophore is attached as a side group to the peptide. In this manner, multiple fluorophores can be attached as side groups to the peptide chain, such as in a manner similar to FIG. 1B.

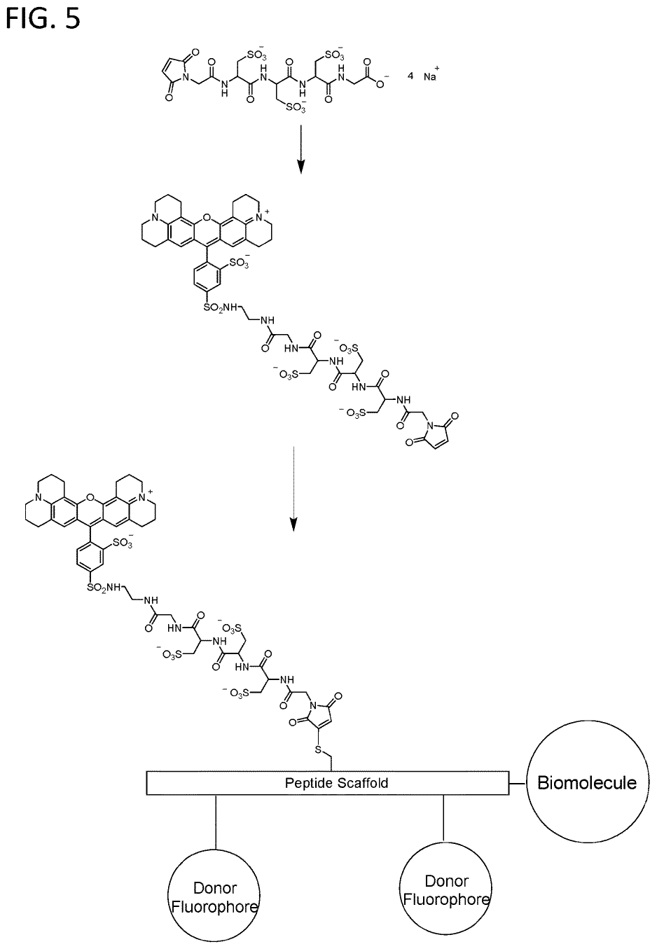

FIG. 5 also shows an embodiment similar to the arrangement of elements in FIG. 1B. Specifically, FIG. 5 shows how a peptide with a chemoselective maleimide group can be covalently bonded to a Texas Red type fluorophore. Afterwards, the peptide-fluorophore compound can be bonded to a peptide scaffold that includes two donor dyes and a biomolecule, e.g., a specific binding member. As such, the peptide with sulfonate groups increases water solubility but also links the Texas Red type acceptor chromophore to the other sections of the tandem dye.

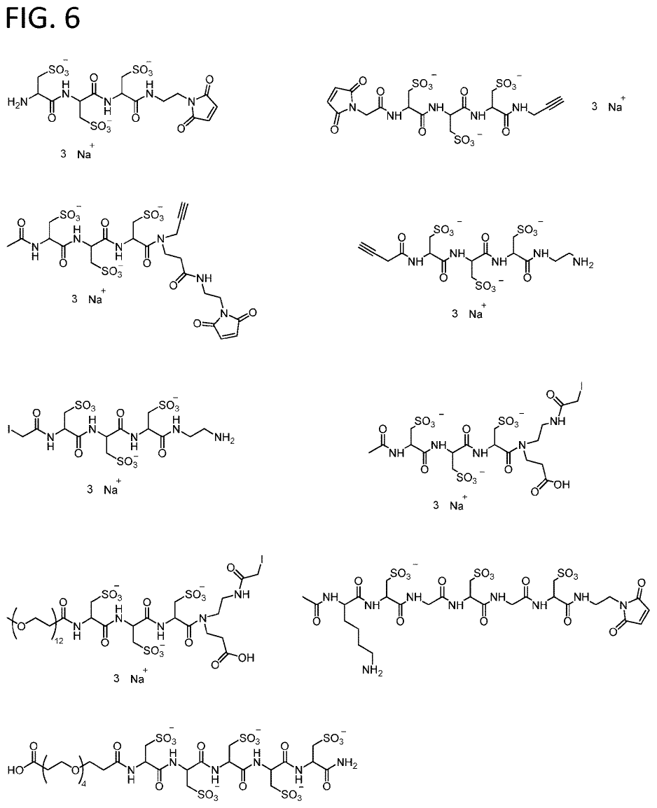

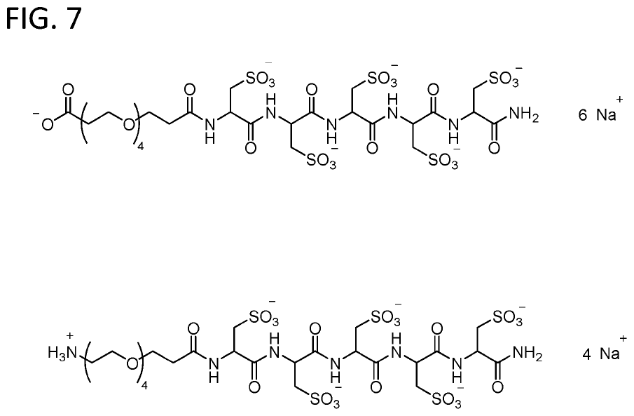

Also provided are further embodiments of water solubilizing peptide groups in FIGS. 6 and 7. Such peptides include various combinations of chemoselective functional groups (e.g., iodo, maleimide, amino, alkyne, carboxylic acid), optional branching points, and optional polyethylene glycol units.

Notwithstanding the appended claims, the disclosure is also defined by the following clauses:

1. A dye comprising:
a fluorophore; and
a water-solubilizing peptide comprising one or more sulfonate groups.

2. The dye of clause 1, wherein the water-solubilizing peptide comprises 2 to 200 amino acid residues.

3. The dye of clause 1 or 2, wherein the water-solubilizing peptide comprises a cysteic acid residue.

4. The dye of clause 3, wherein the water-solubilizing peptide comprises one or more non-cysteic acid residues.

5. The dye of clause 4, wherein the water-solubilizing peptide comprises five or more non-cysteic acid residues.

6. The dye of any one of clauses 1-5, wherein the peptide has formula (I):

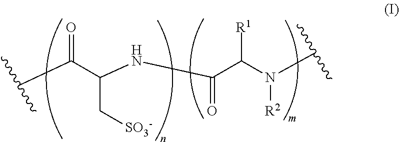

wherein:
each R¹ and R² is independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl, alkoxy, substituted alkoxy, amino, azido, carboxy, cyano, ether, halo, hydroxy, nitro, thiol, thioether, thioketo, borate, —SO₂, and —SO₃—, provided that R¹ and R² can together with the atoms to which they are attached form a heterocyclic group; and
n is an integer ranging from 1 to 200 and m is an integer ranging from 0 to 200, provided that n+m is 2 or more.

7. The dye of clause 6, wherein each R¹ is independently H, alkyl, substituted alkyl, heterocycle, or substituted heterocycle.

8. The dye of any one of clauses 6-7, wherein R² is H.

9. The dye of clause 6, wherein the peptide has formula (II)

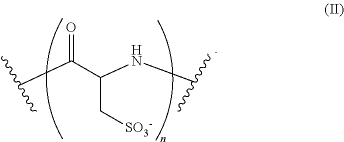

10. The dye of any one of clauses 6-9, wherein n is an integer ranging from 2 to 20.

11. The dye of any one of clauses 1-10, wherein the dye comprises an organic dye.

12. The dye of clause 11, wherein the organic dye is selected from the group consisting of cyanine dyes, rhodamine dyes, xanthene dyes, coumarin dyes, polymethines, pyrenes, dipyrromethene borondifluorides, napthalimides, thiazine dyes, and acridine dyes.

13. The dye of any one of clauses 11-12, wherein the water-solubilizing peptide is bound to the organic dye.

14. The dye of any one of the preceding clauses, further comprising a non-conjugated polymeric backbone comprising non-conjugated repeat units.

15. The dye of clause 14, wherein the non-conjugated repeat units comprise a plurality of amino acid residues.

16. The dye of any one of clauses 14-15, wherein the dye comprises an organic dye bound to the non-conjugated polymeric backbone.

17. The dye of any one of clauses 14-16, wherein the water-solubilizing peptide is bound to the non-conjugated polymeric backbone.

18. The dye of any one of clauses 14-16, wherein the water-solubilizing peptide is bound to the organic dye.

19. The dye of any one of clauses 14-16, wherein the water-solubilizing peptide is a part of the non-conjugated repeat units of the non-conjugated polymeric backbone.

20. The dye of any one of clauses 1-10, wherein the dye comprises a conjugated polymer.

21. The dye of clause 20, wherein the water-solubilizing peptide is bound to the conjugated polymer.

22. The dye of clause 20, wherein the water-solubilizing peptide is bound to an organic dye bound to the conjugated polymer.

23. A tandem dye comprising:
a donor fluorophore;
an acceptor fluorophore; and
a water-solubilizing peptide comprising one or more sulfonate groups.

24. The tandem dye of clause 23, wherein the at least one of the donor and acceptor fluorophore comprises an organic dye.

25. The tandem dye of clause 24, wherein the organic dye is selected from the group consisting of cyanine dyes, rhodamine dyes, xanthene dyes, coumarin dyes, polymethines, pyrenes, dipyrromethene borondifluorides, napthalimides, thiazine dyes, and acridine dyes.

26. The tandem dye of any one of clauses 23-25, further comprising a non-conjugated polymeric backbone comprising non-conjugated repeat units.

27. The tandem dye of clause 26, wherein the non-conjugated repeat units comprise a plurality of amino acid residues.

28. The tandem dye of any one of clauses 26-27, wherein the both the donor fluorophore and acceptor fluorophore comprise an organic dye.

29. The tandem dye of any one of clauses 26-28, wherein the water-solubilizing peptide is bound to the non-conjugated polymeric backbone.

30. The tandem dye of any one of clauses 26-28, wherein the water-solubilizing peptide is a pendant group bound to the donor fluorophore.

31. The tandem dye of any one of clauses 26-28, wherein the water-solubilizing peptide a linker bound to the donor fluorophore and the non-conjugated polymeric backbone.

32. The tandem dye of any one of clauses 26-28, wherein the water-solubilizing peptide is a pendant group bound to the acceptor fluorophore.

33. The tandem dye of any one of clauses 26-28, wherein the water-solubilizing peptide a linker bound to the acceptor fluorophore and the non-conjugated polymeric backbone.

34. The tandem dye of any one of clauses 26-28, wherein the peptide is a part of the non-conjugated repeat units of the non-conjugated polymeric backbone.

35. The tandem dye of any one of clauses 23-25, wherein the donor chromophore comprises a conjugated polymer.

36. The tandem dye of clause 35, wherein the water-solubilizing peptide is a pendant group bound to the donor fluorophore.

37. The tandem dye of clause 35, wherein the water-solubilizing peptide a linker bound to the donor fluorophore and the conjugated polymer.

38. The tandem dye of clause 35, wherein the water-solubilizing peptide is a pendant group bound to the acceptor fluorophore.

39. The tandem dye of clause 35, wherein the water-solubilizing peptide a linker bound to the acceptor fluorophore and the conjugated polymer.

40. A labeled specific binding member comprising:
a dye of any one of clauses 1-22 or a tandem dye of any one of clauses 23-39; and
a specific binding member.

41. The labeled specific binding member of clause 40, wherein the water-solubilizing peptide is linker bound to the specific binding member.

42. A method of evaluating a sample for presence of a target analyte, the method comprising:
(a) contacting the sample with a labeled specific binding member of any one of clauses 40-41 that specifically binds the target analyte to produce a labeled sample; and
(b) assaying the labeled composition for the presence of a labelled specific binding member-target analyte binding complex to evaluate whether the target analyte is present in the sample.

43. A method of labeling a target molecule, the method comprising:
contacting the target molecule with a dye of any one of clauses 1-22 or a tandem dye of any one of clauses 23-39 to covalently bond the target molecule to the conjugation tag, thereby producing the labelled target molecule.

44. The method of clause 43, wherein the target molecule is an antibody or a fragment thereof.

45. A kit comprising:
a dye of any one of clauses 1-22, a tandem dye of any one of clauses 23-39, a labeled specific binding member of any one of clauses 40-41, or a combination thereof; and
a container.

46. A method of increasing the solubility of a compound with a water-solubilizing peptide comprising a sulfonate group, the method comprising:
bonding the water-solubilizing peptide to the compound.

47. The method of clause 46, wherein the compound is a dye comprising a fluorophore or a tandem dye comprising a donor fluorophore and an acceptor fluorophore.

48. The method of clause 46, wherein the compound is a specific binding member or a labeled specific binding member.

49. The method of clause 46, wherein the compound is an active pharmaceutical ingredient (API).

50. The method of clause 46, wherein the compound is selected from the group consisting of a pharmaceutically acceptable excipient, a pharmaceutically acceptable diluent, a pharmaceutically acceptable carrier, and a pharmaceutically acceptable adjuvant.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A dye comprising:
a fluorophore; and
a water-solubilizing peptide comprising one or more sulfonate groups.

2. The dye of claim 1, wherein the water-solubilizing peptide comprises a cysteic acid residue.

3. The dye of claim 2, wherein the water-solubilizing peptide comprises one or more non-cysteic acid residues.

4. The dye of claim 3, wherein the water-solubilizing peptide comprises five or more non-cysteic acid residues.

5. The dye of any one of claims 1-4, wherein the peptide has formula (I): 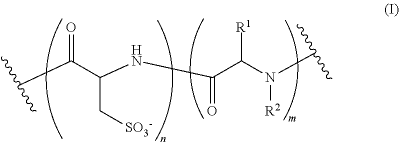
wherein:
each R¹ and R² is independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl, alkoxy, substituted alkoxy, amino, azido, carboxy, cyano, ether, halo, hydroxy, nitro, thiol, thioether, thioketo, borate, —SO₂, and —SO₃—, provided that R¹ and R² can together with the atoms to which they are attached form a heterocyclic group; and
n is an integer ranging from 1 to 200 and m is an integer ranging from 0 to 200, provided that n+m is 2 or more.

6. The dye of claim 5, wherein each R¹ is independently H, alkyl, substituted alkyl, heterocycle, or substituted heterocycle.

7. The dye of claim 5, wherein the peptide has formula (II)

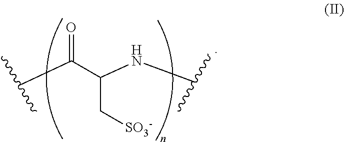

8. The dye of any one of the preceding claims, further comprising a non-conjugated polymeric backbone comprising non-conjugated repeat units.

9. The dye of claim 8, wherein the non-conjugated repeat units comprise a plurality of amino acid residues.

10. The dye of any one of claims 1-7, wherein the dye comprises a conjugated polymer.

11. A tandem dye comprising:
a donor fluorophore;
an acceptor fluorophore; and
a water-solubilizing peptide comprising one or more sulfonate groups.

12. A labeled specific binding member comprising:
a dye of any one of claims 1-10 or a tandem dye of claim 11; and
a specific binding member.

13. A method of evaluating a sample for presence of a target analyte, the method comprising:
(a) contacting the sample with a labeled specific binding member of claim 12 that specifically binds the target analyte to produce a labeled sample; and
(b) assaying the labeled composition for the presence of a labelled specific binding member-target analyte binding complex to evaluate whether the target analyte is present in the sample.

14. A method of labeling a target molecule, the method comprising:
contacting the target molecule with a dye of any one of claims 1-10 or a tandem dye of claim 11 to covalently bond the target molecule to the conjugation tag, thereby producing the labelled target molecule.

15. A kit comprising:
a dye of any one of claims 1-10, a tandem dye of claim 11, a labeled specific binding member of claim 12, or a combination thereof; and
a container.
